(19) United States
(12) Patent Application Publication
(10) Pub. No.: US 2021/0060163 A1
(43) Pub. Date: Mar. 4, 2021

(54) VACCINE FOR TREATMENT OF CANCER AND METHOD OF MAKING BY STRESS REPROGRAMMING

(71) Applicant: VCell Therapeutics, Inc., Hanover, MD (US)

(72) Inventors: Charles A. Vacanti, Naples, FL (US); Martin P. Vacanti, Manhattan, KS (US)

(21) Appl. No.: 17/011,239

(22) Filed: Sep. 3, 2020

Related U.S. Application Data
(60) Provisional application No. 62/895,758, filed on Sep. 4, 2019.

(51) Int. Cl. A61K 39/395; A61K 39/00; A61K 9/00; A61P 35/00

(52) U.S. Cl. CPC A61K 39/39558; A61P 35/00; A61K 9/0019; A61K 39/0011

(57) ABSTRACT

A method has been developed to enhance the efficacy of cancer vaccines by activating the immune system against a greater variety of antigens expressed in the tumor cells. In this modification, the vaccine is created against not only the more mature cancer cells, but also cancer stem cells (CSCs), that act as tumor propagating cells, and can also be made against as the more mature progeny of the CSCs that are normally present within the malignant tumors in numbers which are too low to effectively manufacture a vaccine against their antigens, but which are responsible for recurrence of the malignant tumor. These include pluripotent and stem cells induced from cells in a tumor biopsy by exposure to stress inducing agents that cause the cells to almost die, thereby causing cells to de-differentiate. The method greatly increases the variety of the tumor antigens at which the vaccine is targeted.

### CROSS-REFERENCE TO RELATED APPLICATIONS

This application claims the benefit of and priority to U.S. Provisional Application No. 62/895,758, entitled “Vaccine For Treatment of Cancer and Method of Making By Stress Reprogramming”, filed in the United States Patent and Trademark Office on Sep. 4, 2019, incorporated herein by reference in its entirety.

### FIELD OF THE INVENTION

The invention is generally in the field of modified cell vaccines for cancer, and more specifically a formulation of pluripotent cancer cells formed by stress inducement of the cancer cells, which can be used to induce an immune response to the pluripotent cancer cells.

### BACKGROUND OF THE INVENTION

Cancer vaccines have not been very successful in the treatment of cancers. There are a number of theories, but the majority view now is that cancers contain pluripotent cells that do not express the same antigens as the differentiated cancer cells. As a result, treatments targeted to the cancer antigens do not target or kill the pluripotent cells, resulting in a reoccurrence of the cancer following cessation of treatment as the pluripotent cells proliferate and differentiate to form the cancer.

Ideally one would isolate the pluripotent cells and target the therapy against both the differentiated cells as well as the pluripotent cells. This is extremely difficult, however, since the markers characteristic of the differentiated cancer cells are not always present on the pluripotent cells and the number of pluripotent (or cancer stem cells, CSCs) is very small relative to the number of cancer cells.

It is therefore an object of the present invention to provide cancer stem cells to create an immune response against the tumors which develop from the cancer stem cells.

It is another object of the present invention to provide an efficient method for isolating cancer stem cells from cancer tissue, which can be obtained by biopsy.

It is still another object of the present invention to provide an improved method to induce pluripotency in differentiated cells without introduction of genes into the cells.

It is a still further object of the invention to provide a method and the resulting pluripotent cells, or fragments thereof, for use as a vaccine for the cancer.

It is another object of the present invention to provide a vaccine for inducing an immune response to pluripotent cancer cells, and cells differentiated therefrom, which does not require isolation of the pluripotent cancer cells in the patient or tissue obtained therefrom.

### SUMMARY OF THE INVENTION

A method has been developed to enhance the efficacy of cancer vaccines by activating the immune system against a greater variety of antigens expressed in the tumor cells. In this modification, the vaccine is created against not only the more mature cancer cells, but also cancer stem cells (CSCs), that act as tumor propagating cells, and can also be made against the more mature progeny of the CSCs that are normally present within the malignant tumors in numbers which are too low to effectively manufacture a vaccine against their antigens, but which are responsible for recurrence of the malignant tumor. The method greatly increases the variety of the tumor antigens at which the vaccine is targeted. The method utilizes “stress induced reprogramming” of mature cells to a more primitive state of stemness. This enables two significant improvements to the tumor vaccines that are currently manufactured.

First, the method results in the generation of a sufficient number of cancer stem cells (CSCs), to be added to the lysate used in manufacturing the vaccine. This enables the creation of a vaccine that is not only effective against the antigens expressed by the more mature cells present within the tumors, but consequently also becomes effective against the cancer stem cells (CSCs) present in the tumors in numbers too low to effectively make a vaccine, yet sufficiently high, to cause recurrence and or metastasis of the tumor. Second, in vitro expansion of the “stress reprogrammed” cancer stem cells (CSCs), and allowing them to mature in normal “in vitro” conditions, creates a sufficiently large population of cells that are representative of the entire spectrum of maturity of cells that are present within the tumors, from CSCs to the mature tumor cells.

A cancer vaccine has been developed for use in treating cancers wherein cancer pluripotent or stem cells (jointly referred to as “CSCs” for convenience) are resistant to immunotherapy based on antigens present only in the differentiated cancer cells. These differentiated cells are exposed to a cellular injury that is sublethal, but results in cellular reprogramming to a state of pluripotency. This is achieved by treating the cells with stressing agents to cause the cells to “de-differentiate”, i.e., to become pluripotent (stress reprogramming cells to reverse cell senescence) so that they can be used to immunize the patients against antigens present in the cancer pluripotent or stem cells but not the differentiated cancer cells.

Cellular reprogramming is a process where the epigenetics of a cell nucleus changes with a consequent change in gene expression. For example, an adult cell that is not expressing the protein Oct4 is reprogrammed through epigenetic changes so that the gene for Oct4 is now read and expressed. The mechanism of reprogramming is due to remodeling of chromatin due to removal or addition of methyl groups to either or both DNA and histones and to acetylation or de acetylation of histones. The change in the epigenetic structure may open or close the chromatin structure to allow or repress the expression of certain genes. In general, methylation of DNA or histones suppresses gene expression and closes chromatin while demethylation of DNA and or histone opens chromatin. Acetylation of histone may open or close chromatin. As described herein, stress de differentiates cells through re programming of chromatin via changes of the epigenetic state. Consequentially stress dedifferentiates cells by changing the epigenetics, which changes the gene expression of proteins. All cells have the complete set of genes so it is the unique epigenetic state that determines what the cell is expressing and what the cell is not expressing.

Useful stressing agents include chemical injury by acid exposure, exposure to inflammasomes or ATP, and mechanical injury by electroporation, ultrasonification, trituration, and agitation. Best results are obtained with the combination of mechanical and chemical injury. For example, pluripotency can be induced by agitation at 750 RPMs (or cycles per minute) for 30 minutes in sphere media with ATP in an amount that causes a low pH and activation of inflammasomes.

The SCS induced by cellular reprogramming can be used as a vaccine, or to make antibodies to the antigens present on the SCS as well as on the cancer cells which are then administered alone or with other anti-proliferative agents to kill the cancers. The agents used to treat the cancer patients may also be a vaccine made with the pluripotent cells to induce an immune response to the non-fully differentiated cancer cells, and/or to make antibody (including humanized antibody, antibody fragment, and derivatives thereof) to the non-fully differentiated cancer cells, which are then administered prior to, at the time of or after surgery and/or chemotherapy. These cells can also be used to test for sensitivity to conventional chemotherapeutic agents to determine which would be most effective in treating the patient.

The cells can be obtained during a biopsy of the cancer patient. It is not necessary to separate out the differentiated cells from the pluripotent or undifferentiated cells. The tissue or dissociated cells are exposed to an effective amount of stressing agents, which result in the death of many differentiated cells or the de-differentiation of others. Useful stressing agents include freezing, pH less than 6, more preferably less than 5.8, ATP, and mechanical disruption, for example, by the turbulence associated with trituration.

Methods for inducing pluripotency are described in WO2015/143125. An improved method of inducing pluripotency has been developed. There are several differences between the original protocol that had a success rate of between 15 and 20%, and the improved protocols that increase the success rate to between 85 and 100%. In the original protocol, the cells were washed, centrifuged, and then the supernatant over the resulting cell pellet was removed and the cells were resuspended in a solution of HBSS (HBSS Ca⁺Mg⁺ Free: Gibco 14170-112). ATP (Adenosine 5′ Triphosphate Disodium Salt Hydrate—Sigma A2383) was then very slowly added to the cell suspension while monitoring the pH, until the pH of the cell suspension was less than 5.0. The cell suspension (in HBSS) was then triturated through a series of reduced bore pipettes with the final, smallest pipet, having an internal diameter of 50 to 70 μm. The pipettes used for trituration were first “pre-coated” with media to discourage adherence of the cells to the pipettes during stress treatment. The “stress treated” cells were then placed in vitro, into specially coated, non adherent tissue culture dishes.

The “stress treatment” methods have now been standardized to reduce variability. In this process, several unnecessary steps have been eliminated, while additional, important steps have been added. In a preferred embodiment, the cells are initially placed directly into sphere media (DMEM/F12 with 1% Antibiotic and 2% B27 Gibco 12587-010 plus the supplements: b-FGF (20 ng/ml), EGF (20 ng/ml), heparin (0.2%, Stem Cell Technologies 07980) without washing or centrifuging prior to performing the stress treatments. Cells put into sphere media at a concentration of 0.1 million to 5 million cells/cc being optimal. ATP in a concentration of 200 micromolar is added to the cell suspension in the amount of 100 μl per 3 cc of cells treated (or 33 μl/cc). The resultant cell suspension, containing the ATP, is then repeatedly injected into and then withdrawn from a 20 ml conical tube, open to air, using a 10 ml syringe connected to standard size orifices (biosilicate microcapillary tubes, or standard needles) having internal diameters between 200 and 500 μl. Under a sterile hood using either standard needles or biosilicate microcapillary tubes, of the following sizes: 21 gauge (I.D.=500 ul), 23 gauge (I.D.=340 ul), 25 gauge (I.D.=260 ul), or 27 gauge (I.D.=210 ul), that are bent without kinking” to enable an unobstructed injection and withdrawal of the cell suspension for 25 minutes, open to air, under a sterile hood. The following standard size biosilicate microcapillary tubes, of the following sizes to also work well, utilizing the same programmed syringe pump system. 5, 10, and 50 μl, biosilicate (glass) microcapillary tubes, which have internal diameters that are comparable to the internal diameters of the above standard size needles that we found to be useful, being 330 μl, 480 μl, and 960 μl respectively. The capillary tubes are connected to the syringes containing the cell suspension and the ATP, using an 18 gauge needle and a short length silastic microtube, to add flexibility to place the microtubes directly into the 20 ml conical tubes, open to air. The trituration process, (repeated injection and withdrawal) is performed using an automated programmable syringe pump. The rate of injection and withdraw varies with number of “cc”s that are held in the 10 ml syringe. An average rate for a suspension containing 6 ml (2 cell suspension aliquots), is about 1 minute/cycle×25 cycles.

Cells in humans range from about 7 microns (red blood cells) to over 100 microns (reactive macrophages). A neuron can measure in the centimeters. A skilled lab tech can fire polish a glass pasteur pipette down to 15 microns in diameter at the tip. World precision instruments has a pipette with a tip diameter of 0.5 microns. MV.

Rather than placing the now “stress treated” cell suspension into low adherence tissue culture dishes, the cell suspension is placed into normal adherence tissue culture dishes, in aliquots of 3 mL of treated cells per 100 mm tissue culture dish. 10 cc of additional sphere media is then added to each dish. After stress treatment, the number of cells remaining is counted. Successful stress treatments are generally associated with approximately a 50% decrease in the total number of viable cells remaining after the treatment.

The next significant modification is that instead of gently pipetting the cells suspensions in each culture dish on a daily basis for a week, after 24 to 36 hours in vitro, the “injured” cells that remain within each tissue culture dish are allowed to attach to the bottom of the dish, and the supernatant over the attached cells, including the associated “floating debris” are removed, discarded, and replaced with 10 ml of fresh sphere media. Up to 2 ml of fresh media is added up to once per week until floating spheres appear in each tissue culture dish, unless the media becomes acidotic as reflected by a color metric change to yellow, in the otherwise, normally pink media. This is in contrast to the previous protocol in which the media was changed much more frequently.

Another improvement is the creation of floating spheres containing “stress reprogrammed” cells, by exposure of an aliquot of the cells to be reprogrammed, suspended in 100 μl of sphere media, without ATP, to a standard dose of electroporation to create small holes in the cells. This is done in the absence of the buffers that are normally added to the solution during standard electroporation to promote repair of the holes created in the cells, which in the case of “stress treatments”, is undesirable.

### DETAILED DESCRIPTION OF THE INVENTION

### I. Definitions

Stem cells are special cells that have the ability to develop into many different cell types. The term generally refers to progenitor cells which can turn into any cells of a single particular germ layer; that is either endoderm, mesoderm, or ectoderm.

Pluripotent Cells are stem cells that have the potential to turn into any cells representative of any of the three germ layers; that is, they cross germ layers, and can turn into any cell type normally found in the body.

As used herein, a stressing agent is any agent that results in the creation of an environment that is extremely hostile to cells, that normally results in significant injury or death to cells exposed to such an agent, i.e., a lethal or sub lethal environment, to cells exposed to such an environment. The hostile environment can be created by any stressing agent, including chemicals, mechanical perturbations, electrical exposure, radiation, pH, ultrasound or application of any external condition or force that is hostile to living cells.

A vaccine is a substance used to stimulate the production of antibodies and elicit immunity against one or several Antigens (surface proteins) expressed in specific disease processes. In this case, the Antigens are surface proteins expressed by cells present within a malignant tumor. The vaccine can be prepared from the causative agent of a disease (in this case, the tumor itself, or the cells contained within the tumor), its products, or a synthetic substitute, treated to act as an antigen without inducing the disease. The antigens are substances on the surface of cells that are not normally part of the body. The immune system is stimulated by the vaccine to attack the antigens, usually getting rid of them. This leaves the immune system with a “memory” that helps it respond to those antigens in the future. Cancer treatment vaccines boost the immune system's ability to recognize and destroy antigens present on the cancer cells. Cancer cells often have certain molecules called cancer-specific antigens on their surface that healthy cells do not have. When these molecules used to manufacture a vaccine, the molecules act as antigens. The vaccine then stimulates the immune system to recognize and destroy cancer cells that have these molecules on their surface. Many cancer vaccines also contain adjuvants, which are substances that may help strengthen the immune response. In this embodiment, the cancer vaccines are manufactured to target the surface antigens present individual patient's tumor. This type of vaccine is produced from the cells acquired form the person's tumor sample, and then stress treated to ultimately generate large populations of cancer stem cells and all of their progeny including the more mature cancer cells. This enables the manufacture of an effective vaccine from a small biopsy of the tumor, rather than necessitating surgery to get a large enough sample of the tumor to create the vaccine, as is the practice with other cancer vaccines.

An immune response is the body's response caused by its immune system being activated by antigens. In one embodiment, the immune system is activated to destroy all of the cells, that are not recognized as “self”, that express the surface antigens (proteins) of the cancer stem cells and all their progeny including all of the immature and more mature cancer cells that are present within the tumor.

As used herein, the term “select”, when used in reference to a cell or population of cells, refers to choosing, separating, segregating, and/or selectively propagating one or more cells having a desired characteristic. The term “select” as used herein does not necessarily imply that cells without the desired characteristic are unable to propagate in the provided conditions.

Sphere media is DMEM/F12 with 1% Antibiotic and 2% B27 Gibco 12587-010 plus the supplements: b-FGF (20 ng/ml), EGF (20 ng/ml), heparin (0.2%, Stem Cell Technologies 07980).

As used herein, “maintain” refers to continuing the viability of a cell or population of cells. A maintained population will have a number of metabolically active cells. The number of these cells can be roughly stable over a period of at least one day or can grow.

As used herein, a “detectable level” refers to a level of a substance or activity in a sample that allows the amount of the substance or activity to be distinguished from a reference level, e.g. the level of substance or activity in a cell that has not been exposed to a stress. In some embodiments, a detectable level can be a level at least 10% greater than a reference level, e.g. 10% greater, 20% greater, 50% greater, 100% greater, 200% greater, or 300% or greater.

The term “statistically significant” or “significantly” refers to statistical significance and generally means a two standard deviation (2SD) difference above or below a reference, e.g. a concentration or abundance of a marker, e.g. a stem cell marker or differentiation marker. The term refers to statistical evidence that there is a difference. It is defined as the probability of making a decision to reject the null hypothesis when the null hypothesis is actually true. The decision is often made using the p-value.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of health, delay or slowing of the disease progression, and amelioration or palliation of symptoms. Treatment can also include the subject surviving beyond when mortality would be expected statistically.

As used herein, the term “administering,” refers to the placement of a pluripotent cell produced according to the methods described herein and/or the at least partially differentiated progeny of such a pluripotent cell into a subject by a method or route which results in at least partial localization of the cells at a desired site. A pharmaceutical composition comprising a pluripotent cell produced according to the methods described herein and/or the at least partially differentiated progeny of such a pluripotent cell can be administered by any appropriate route which results in an effective treatment in the subject.

### II. Compositions and Methods of Making

Methods to make modified cell vaccines for the treatment of cancer have been developed based on the generation of “stress reprogrammed” cancer stem cells (CSCs) that can be used to induce an immune response to cancer stem cells, and their progeny, that are known to exist within malignant tumors and are believed to be responsible for metastasis or recurrence of the tumors in spite of therapies that would otherwise have killed the more mature cancer cells also contained within the tumor. Efficacy of the vaccine is enhanced by activating the immune system against a greater variety of antigens expressed in the tumor cells. The vaccine is created against not only the more mature cancer cells, but also cancer stem cells (CSCs), that act as tumor propagating cells, as well as against the more mature progeny of the CSCs that are normally present within the malignant tumors in numbers which are too low to effectively manufacture a vaccine against their antigens, but which are responsible for recurrence of the malignant tumors. This method greatly increases the variety of the tumor antigens at which the vaccine is targeted. Tumor antigens may be proteins, peptides or glycoproteins. Tumor tissue is obtained by biopsy or excision of the original tumor or a metastatic focus.

L B Driscoll, Nature Communications, 7 Feb. 2020 volv11, “APOBEC3B mediated corruption of the tumor cell immunopeptide induces heteroclitic neopeptides for cancer immunotherapy” shows that treating a tumor with a super mutagen, makes many more protein antigens resulting in a more effective tumor vaccine, and that cell adhesiveness serves as a biophysical marker for metastatic potential. Pranjali Beri, Cancer Res. 2020 shows that the less adhesive tumor cells are the more they metastasize. Studies in which a patient's senescent glioblastoma cells were stressed in culture showed that they converted to extremely malignant looking tumorspheres with numerous irregular mitotic figures indicating rapid proliferation, and that they were no longer senescent. The tumorspheres did not attach to the culture plates, indicating that they had lost all their adhesive capabilities and were highly malignant. They did continue to increase in size, most likely do to their high proliferation rate. As they increased in size, the peripheral cells would differentiate relative to the center stem cells. If the center cells necrosed, the tumor antigens would still be present. The tumor cell numbers can be calculated by measuring the total volume of the tumorspheres. These are useful cells or sources of antigens to use in vaccines.

The CSCs are obtained using “stress induced reprogramming” of mature cells into a more primitive state of differentiation (i.e., dedifferentiates the cancer cells). This enables two significant improvements to the tumor vaccines that are currently manufactured:

(1) It results in the generation of a sufficient number of cancer stem cells (CSCs), to be added to the lysate used in manufacturing the vaccine. This enables the creation of a vaccine that is not only effective against the antigens expressed by the more mature cells present within the tumors, but consequently also becomes effective against the cancer stem cells (CSCs) present in the tumors in numbers too low to effectively make a vaccine, yet sufficiently high, to cause recurrence and or metastasis of the tumor.

(2) In vitro expansion of the “stress reprogrammed” cancer stem cells (CSCs), and allowing them to mature in normal “in vitro” conditions, creates a large population of cells that are representative of the entire spectrum of maturity of cells that are present within the tumors, from CSCs to the mature tumor cells, which can then be used for vaccination or generation of antibodies to kill the tumors.

The addition of cancer stem cells and their somewhat more mature progeny to the vaccine based on a very small number of antigens present on the differentiated cancer greatly increases the variety of foreign tumor antigens needed to elicit a much more effective immune response to malignant tumors.

These cells should also be useful in the development and screening of therapies for the treatment of these cancers. The CSCs and slightly matured CSCs can be used as a source of antigen, to study mechanisms and actions and potential targets for chemotherapy or immunotherapy, both humoral and cell mediated immunotherapy.

Critical features of the methods of stress inducing pluripotency in the cells include the application of chemical stressing agents including low pH, ATP, and mechanical stresses such as trituration or freezing that damage the cell wall integrity. Care should be taken to use sublethal amounts and conditions.

In a preferred embodiment, the targeted cells in suspension are exposed to a low pH solution containing ATP at a 0.20 millimolar concentration (110 mg/ml), resulting in an acidic solution with a pH of less than 3.5. Then, 33 μl of this solution is added to each 1 ml of the cell suspension to be stress treated. This results in a final cell suspension containing 0.363 mg/ml of ATP, or 363 ng of ATP/ml. The cell suspension is exposed, while being agitated or triturated, to the ATP in solution for 30 minutes. The initial addition of the ATP to the cell suspension raises the pH of the entire suspension to approximately 5.5, and then the agitation or trituration causes the pH to increase over the 30 minutes of treatment, to neutral pH or pH 7.0.

It is believed that the ATP solution acts as an inflammasome inducer, an inflammasome, being a multiprotein oligomer that activates an inflammatory response. This process mimics the normal healing process. After significant injuries, an inflammatory response is initiated which removes the injured cells to enable initiation of wound healing. This inflammatory response not only results in the death of the most severely injured cells, but equally importantly results in sub-lethal injury of cells adjacent to the injured area. It is the cells that sustain sub-lethal injuries that are reprogrammed to a level of stemness, that actually replace the fatally injured cells. The process using pH, ATP and/or mechanical stimuli exposes the cancer cells to sublethal injuries, stress reprogramming them in a manner that results in the formation of spheres containing mixed populations of stress reprogrammed stem cells (CSCs), or, in the case of glioblastomas, brain tumor propagating cells (BTPCs), which are then utilized to enhance the efficacy of vaccine made against the differentiated tumor cells, the combination of stress reprogrammed stem cells in combination with differentiated cancer cells activating the immune response against all of the cells in the glioblastoma, including those responsible for metastasis and recurrence of the malignancy.

### A. Induced Pluripotent Cancer Cells

Cells can be obtained from tumors in a patient or from established cells lines. Cancer cells can be brain tumors, especially glial blastoma tumors, breast cancers, lung cancers, or other types of tumors where cancer stem cells have been show to play a role in resistance to chemotherapy or radiation.

The primary types of cancer include:

Carcinomas: cancer that begins in the skin or in tissues that line or cover internal organs. There are different subtypes, including adenocarcinoma, basal cell carcinoma, squamous cell carcinoma and transitional cell carcinoma.

Sarcomas: cancer that begins in the connective or supportive tissues such as bone, cartilage, fat, muscle or blood vessels.

Leukemias: cancer that starts in blood forming tissue such as the bone marrow and causes abnormal blood cells to be produced and go into the blood.

Lymphomas and myelomas: cancers that begin in the cells of the immune system.

Brain and spinal cord cancers, known as central nervous system cancers.

### B. Vaccines

Methods to make cancer vaccines are known and described in the literature. See, for example, Tagliamonte, et al. Hum Vaccin Immunother. 10(11): 3332-3346 (2014); See also Taglilamonte, et al. Clin. Vaccine Immunol. 18(1): 23-34 (2011).

Process to make a vaccine from only a small biopsy.

In normal situations, a biopsy of the tumor is first obtained.

Cells from the biopsy specimen are allowed to shed from the biopsy in media in standard cell culture conditions for 10 days “in Vitro”.

Autologous monocytes are acquired from the patient.

Monocyte-derived dendritic cells are generated in vitro from peripheral blood mononuclear cell (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to immature dendritic cells (iDCs) in about a week.

The resultant dendritic cells have a very large surface area to volume ratio.

The dendritic cells are then exposed to the lysate made in step 2.

Immature dendritic cells phagocytose pathogens and degrade their proteins into small pieces and upon maturation present those fragments at their cell surface using MHC molecules.

Once they have come into contact with a presentable antigen, they become activated into mature dendritic cells and begin to migrate to the lymph node.

They also “nibble” on autologous cells, and then come to recognize them as “self” so that autologous cells are not attacked in the process.

Once the dendritic cells are activated by the foreign antigens, they migrate to the lymph nodes where they interact with T cells and B cells to initiate and shape the immune response. It is felt that the greater the variety of antigens present, the more effective the vaccine will be.

Vaccines represent a strategic successful tool used to prevent or contain diseases with high morbidity and/or mortality. However, while vaccines have proven to be effective in combating pathogenic microorganisms, based on the immune recognition of these foreign antigens, vaccines aimed at inducing effective antitumor activity are still unsatisfactory. Nevertheless, the effectiveness of the two licensed cancer-preventive vaccines targeting tumor-associated viral agents (anti-HBV [hepatitis B virus], to prevent HBV-associated hepatocellular carcinoma, and anti-HPV [human papillomavirus], to prevent HPV-associated cervical carcinoma), along with the recent FDA approval of SIPULEUCEL-T (for the therapeutic treatment of prostate cancer), represents a significant advancement in the field of cancer vaccines and a boost for new studies in the field. Specific active immunotherapies based on anticancer vaccines represent, indeed, a field in continuous evolution and expansion. Significant improvements may result from the selection of the appropriate tumor-specific target antigen (to overcome the peripheral immune tolerance) and/or the development of immunization strategies effective at inducing a protective immune response.

### C. Combination Therapies Including Radiation and Chemotherapeutic Agents

This vaccine therapy can be combined with any therapy that is currently combined with vaccine treatments. The improvements do not hinder the efficacy of any currently effective combination of therapies with that of a vaccine.

There are many types of cancer treatment. The types of treatment depend on the type of cancer and how advanced it is. Some people with cancer will have only one treatment, but most people have a combination of treatments, such as surgery with chemotherapy and/or radiation therapy, have immunotherapy, targeted therapy, or hormone therapy.

Radiation therapy is a type of cancer treatment that uses high doses of radiation to kill cancer cells and shrink tumors. Learn about the types of radiation, why side effects happen, which ones you might have, and more.

Chemotherapy is a type of cancer treatment that uses drugs to kill cancer cells. Learn how chemotherapy works against cancer, why it causes side effects, and how it is used with other cancer treatments.

Immunotherapy is a type of treatment that helps your immune system fight cancer.

Targeted therapy is a type of cancer treatment that targets the changes in cancer cells that help them grow, divide, and spread.

Hormone therapy is a treatment that slows or stops the growth of breast and prostate cancers that use hormones to grow.

Stem cell transplants are procedures that restore blood-forming stem cells in cancer patients who have had theirs destroyed by very high doses of chemotherapy or radiation therapy.

### III. Methods of Inducing Pluripotent Cells

The cells can be obtained during a biopsy of the cancer patient. It is not necessary to separate out the differentiated cells from the pluripotent or undifferentiated cells. The tissue or dissociated cells are exposed to an effective amount of one or more stressing agents until differentiated cells die or de-differentiate. Useful stressing agents include freezing, pH less than 6, more preferably less than 5.8, ATP, and mechanical disruption, for example, by trituration. Methods for inducing pluripotency are described in WO2015/143125. These methods have been significantly improved and expanded, as described below.

Cells are subjected to stress to induce pluripotency in cells. In some embodiments, the stress results in the loss of about 40%, 50%, or 60-80% of the cytoplasm and/or mitochondria from the cell. In some embodiments, the stress is sufficient to disrupt the cellular membrane of at least 10% of cells exposed to the stress. In some embodiments, selecting cells exhibiting pluripotency comprises selecting cell which are not fatally injured, and consequently retain the ability to adhere to the bottom of the petri dishes.

In some embodiments, the stress comprises exposure of the cell to at least one environmental stimulus selected from: trauma, mechanical stimuli, chemical exposure, ultrasonic stimulation, oxygen-deprivation, radiation, and exposure to extreme temperatures. In some embodiments, the stress comprises exposing the cell to a pH of from about 4.5 to about 6.0. In some embodiments, the stress comprises exposing the cell to a pH of from about 5.4 to about 5.8. In some embodiments, the cell is exposed for 1 day or less. In some embodiments, the cell is exposed for 1 hour or less. In some embodiments, the cell is exposed for about 30 minutes.

In some embodiments, the exposure to extreme temperatures comprises exposing the cell to temperatures below 35° C. or above 42° C. In some embodiments, the exposure to extreme temperatures comprises exposing the cell to temperatures at, or below freezing or exposure of the cell to temperatures at least about 85° C. In some embodiments, the removal of a portion of the cytoplasm removes at least about 50% of the mitochondria from the cytoplasm. In some embodiments, the removal of cytoplasm or mitochondria removes about 50%-90% of the mitochondria from the cytoplasm. In some embodiments, the removal of cytoplasm or mitochondria removes more than 90% of the mitochondria from the cytoplasm.

An improved method of inducing pluripotency has been developed. There are several differences between the original protocol that had a success rate of between 15 and 20%, and the improved protocols that increase the success rate to between 85 and 100%. In the original patent application, the inventors did not have a complete understanding of the mechanism of the stress reprogramming of the cells.

The applied stresses results in activation of what occurs in the normal wound healing process. While older theories of wound healing attribute the tissue repair to the recruitment of stem cells from distal sites such as bone marrow, or the spleen, or from mysterious stem cell “niches”, normal wound healing after injury occurs as a result of stress reprogramming of injured cells to revert to stem cells that are seriously injured, yet survive the injury; that is; sub lethally injured cells in the area of the injury, or adjacent to the injury that survive, are reprogrammed to become stem cells and repair the injury. By mimicking this process, developed a better understanding of the mechanism of “stress reprogramming” of injured cells to a state of stemness, and were able to develop significant “non obvious” improvement to the previously described methods.

one embodiment, ATP was added to the treated cells suspension, as a potential, very simple energy source for the “stress injured” cells. The ATP solution itself acts as an “inflammasome inducer”, that activates the inflammatory process that was causing the injury to the cells. Consequently, it is the ATP solution itself that can be sufficient as a stress treatment, as are other chemicals normally released during activation of the inflammatory process.

In earlier descriptions in which it was believed that the cells responsible for the formation of spheres containing “stress reprogrammed cells” were contained in the supernatant of the petri dishes of the cultured, treated cells, efforts were made to discourage adherence of the treated cell populations to the bottoms of the culture dishes. It is now known that injured cells that eventually die lose the ability to adhere to the dishes. Consequently, cells are now allowed to attach to the dishes for between several hours and 24 hours. These severely, yet sub lethally injured cells that retain the ability to attach to the dishes, are the cells that result in the formation of spheres containing the stress reprogrammed stem cells.

In the original protocol, the cells were washed, and centrifuged, HBSS, and then the supernatant over the resulting cell pellet was removed. The cells are now initially collected in sphere media where they are stress treated. The cells can be obtained during a biopsy of the cancer patient. It is not necessary to separate out the differentiated cells from the pluripotent or undifferentiated cells. The tissue or dissociated cells are exposed to an effective amount of one or more stressing agents until approximately half or more of the differentiated cells die, leaving the not lethally injured remaining cells that survive the insult to become stress reprogrammed. Useful stressing agents include freezing, pH less than 6, more preferably less than 5.8, the known inflammasome inducer, ATP, and mechanical disruption, for example, by trituration.

Stress can induce the production of pluripotent stem cells from cells without the need to introduce an exogenous gene, a transcript, a protein, a nuclear component or cytoplasm to the cell, or without the need of cell fusion. In some embodiments, the stress induces a reduction in the amount of cytoplasm and/or mitochondria in a cell; triggering a dedifferentiation process and resulting in pluripotent cells. In some embodiments, the stress causes a disruption of the cell membrane, e.g. in at least 10% of the cells exposed to the stress. These pluripotent cells can differentiate into each of the three germ layers (in vitro and/or in vivo).

An improved method of inducing pluripotency has been developed. There are several differences between the original protocol that had a success rate of between 15 and 20%, and the improved protocols that increase the success rate to between 85 and 100%. In the original protocol, the cells were washed, centrifuged, and then the supernatant over the resulting cell pellet was removed and the cells were resuspended in a solution of HBSS (HBSS Ca+Mg+ Free: Gibco 14170-112). ATP (Adenosine 5′ Triphosphate Disodium Salt Hydrate—Sigma A2383) was then very slowly added to the cell suspension while monitoring the pH, until the pH of the cell suspension was less than 5.0. The cell suspension (in HBSS) was then triturated through a series of reduced bore pipettes with the final, smallest pipet, having an internal diameter of 50 to 70 μm. The pipettes used for trituration were first “pre-coated” with media to discourage adherence of the cells to the pipettes during stress treatment. The “stress treated” cells were then placed in vitro, into specially coated, non adherent tissue culture dishes.

The “stress treatment” methods have now been standardized to reduce variability. In this process, several unnecessary steps have been eliminated, while additional, important steps have been added. In the improved protocols, the cells are initially placed directly into the sphere media (DMEM/F12 with 1% Antibiotic and 2% B27 Gibco 12587-010 plus the supplements: b-FGF (20 ng/ml), EGF (20 ng/ml), heparin (0.2%, Stem Cell Technologies 07980) without washing or centrifuging prior to performing the stress treatments. Cells put into sphere media at a concentration of 2-5 million cells/cc is optimal. ATP in a concentration of 200 micromolar is added to the cell suspension in the amount of or 33 μl/cc of treated cells. The resultant cell suspension, containing the inflammasome inducer, ATP, is then repeatedly injected into and then withdrawn from a 20 ml conical tube, open to air, using a 10 ml syringe connected to standard size orifices (biosilicate microcapillary tubes, or standard needles) having internal diameters between 200 and 500 μl. Under a sterile hood using either standard needles or biosilicate microcapillary tubes, of the following sizes: 21 gauge (I.D.=500 ul), 23 gauge (I.D.=340 ul), 25 gauge (I.D.=260 ul), or 27 gauge (I.D.=210 ul), that are bent without kinking” to enable an unobstructed injection and withdrawal of the cell suspension for 25 minutes, open to air, under a sterile hood. The following standard size biosilicate microcapillary tubes, of the following sizes to also work well, utilizing the same programmed syringe pump system. 5, 10, and 50 μl, biosilicate (glass) microcapillary tubes, which have internal diameters that are comparable to the internal diameters of the above standard size needles were found to be useful, being 330 μl, 480 μl, and 960 μl respectively. The capillary tubes are connected to the syringes containing the cell suspension and the ATP, using an 18 gauge needle and a short length silastic microtube, to add flexibility to place the microtubes directly into the 20 ml conical tubes, open to air. The trituration process, (repeated injection and withdrawal), is performed using an automated programmable syringe pump. The rate of injection and withdraw varies with number of “cc”s that are held in the 10 ml syringe. An average rate for a suspension containing 6 ml (2 cell suspension aliquots), is about 1 minute/cycle×25 cycles.

In another embodiment, the cell suspension containing the inflammasome inducing ATP solution is vigorously agitated for 30 minutes at a rate of 500-1000 cycles/minute, without the need for mechanical trituration.

Rather than placing the now “stress treated” cell suspension into low adherence tissue culture dishes, the cell suspension is placed into normal adherence tissue culture dishes, in aliquots of 3 mL of treated cells per 100 mm tissue culture dish for 24 hours during which time, the injured, but still viable cells are allowed to attaché to the bottoms of the Petri dishes, after which time, the non adherent cells are removed with the supernatant, discarded, and replaced with 10-15 ml of fresh sphere media. Previously it was believed that the supernatant containing the non adherent cells also contained the stress reprogrammed cells. It has now been learned that the majority of the spheres composed of “stress reprogrammed cells arise from the sublethally injured cells still retain the ability to attach to the bottoms of the dishes, while the supernatant contains mostly dead cells and debris, but can contain small numbers of stress reprogrammed cells. After removal of the overlying supernatant, 10 cc of fresh sphere media is then added to each dish. After stress treatment, the number of cells remaining is counted. Successful stress treatments are generally associated with approximately a 50% decrease in the total number of viable cells remaining after the treatment.

A significant modification is that instead of gently pipetting the cells suspensions in each culture dish on a daily basis for a week, after 24 to 36 hours in vitro, the “injured” cells that remain within each tissue culture dish are allowed to attach to the bottom of the dish, and the supernatant over the attached cells, including the associated “floating debris” are removed, discarded, and replaced with 10 ml of fresh sphere media. The media is changed once per week until floating spheres appear in each tissue culture dish. This is in contrast to the previous protocol in which the media was changed much more frequently.

Another improvement is the creation of floating spheres containing “stress reprogrammed” cells, by exposure of an aliquot of the cells to be reprogrammed, suspended in 100 μl of sphere media, without ATP, to a standard dose of electroporation to create small holes in the cells. This is done in the absence of the buffers that are normally added to the solution during standard electroporation to promote repair of the holes created in the cells, which in the case of “stress treatments”, is undesirable.

A system for generating a pluripotent cell from a cell, according to the methods described herein, can comprise a container in which the cells are subjected to stress. The container can be suitable for culture of somatic and/or pluripotent cells, as for example, when cells are cultured for days or longer under low oxygen conditions in order to reduce the amount of cytoplasm and/or mitochondria according to the methods described herein. Alternatively, the container can be suitable for stressing the cells, but not for culturing the cells, as for example, when cells are triturated in a device having a narrow aperture for a limited period, e.g. less than 1 hour. Alternatively, cells can be vigorously agitated in sterile conical tubes, as described above. A container can be, for example, a vessel, a tube, a microfluidics device, a pipette, a bioreactor, or a cell culture dish. A container can be maintained in an environment that provides conditions suitable for the culture of somatic and/or pluripotent cells (e.g. contained within an incubator) or in an environment that provides conditions which will cause environmental stress on the cell (e.g. contained within an incubator providing a low oxygen content environment). A container can be configured to provide 1 or more of the environmental stresses described above herein, e.g. 1 stress, 2 stresses, 3 stresses, or more. Containers suitable for manipulation and/or culturing somatic and/or pluripotent cells are well known to one of ordinary skill in the art and are available commercially (e.g. Cat No CLS430597 Sigma-Aldrich; St. Louis, Mo.). In some embodiments, the container is a microfluidics device. In some embodiments, the container is a cell culture dish, flask, conical tube or plate.

In some embodiments, the system includes means for selecting pluripotent cells, such as a FACS system which can select cells expressing a pluripotency marker (e.g. Oct4-GFP) or select by size as described above herein. Methods and devices for selection of cells are well known to one of ordinary skill in the art and are available commercially, e.g. BD FACSARIA SORP.™. coupled with BD LSRII.™. and BD FACSDIVA.™. Software (Cat No. 643629) produced by BD Biosciences; Franklin Lakes, N.J.

The “stress treatment” methods have been standardized to reduce variability. In this process, several unnecessary steps have been eliminated, while additional, important steps have been added. In the improved protocols, the cells are initially placed directly into the sphere media (DMEM/F12 with 1% Antibiotic and 2% B27 Gibco 12587-010 plus the supplements: b-FGF (20 ng/ml), EGF (20 ng/ml), heparin (0.2%, Stem Cell Technologies 07980) without washing or centrifuging prior to performing the stress treatments. Cells put into sphere media at a concentration of 2-5 million cells/cc is optimal. The inflammasome inducer, ATP in a concentration of 200 micromolar is added to the cell suspension in the amount of 33 μl/ml. The resultant cell suspension, containing the ATP, is then repeatedly injected into and then withdrawn from a 20 ml conical tube, open to air, using a 10 ml syringe connected to standard size orifices (biosilicate microcapillary tubes, or standard needles) having internal diameters between 200 and 500 μl. Under a sterile hood using either standard needles or biosilicate microcapillary tubes, of the following sizes: 21 gauge (I.D.=500 ul), 23 gauge (I.D.=340 ul), 25 gauge (I.D.=260 ul), or 27 gauge (I.D.=210 ul), that are bent without kinking” to enable an unobstructed injection and withdrawal of the cell suspension for 25 minutes, open to air, under a sterile hood. The following standard size biosilicate microcapillary tubes, of the following sizes to also work well, utilizing the same programmed syringe pump system. 5, 10, and 50 μl, biosilicate (glass) microcapillary tubes, which have internal diameters that are comparable to the internal diameters of the above standard size needles that were useful, being 330 μl, 480 μl, and 960 μl respectively. The capillary tubes are connected to the syringes containing the cell suspension and the ATP, using an 18 gauge needle and a short length silastic microtube, to add flexibility to place the microtubes directly into the 20 ml conical tubes, open to air. The trituration process, (repeated injection and withdrawal), is performed using an automated programmable syringe pump. The rate of injection and withdraw varies with number of “ml”s that are held in the 10 ml syringe. An average rate for a suspension containing 6 ml (2 cell suspension aliquots), is about 1 minute/cycle×25 cycles.

Rather than placing the now “stress treated” cell suspension into low adherence tissue culture dishes, the cell suspension is placed into normal adherence tissue culture dishes, in aliquots of 3 mL of treated cells per 100 mm tissue culture dish. 10 ml of additional sphere media is then added to each dish. After stress treatment, the number of cells remaining is counted. Successful stress treatments are generally associated with approximately a 50% decrease in the total number of cells remaining after the treatment.

The next significant modification is that instead of gently pipetting the cells suspensions in each culture dish on a daily basis for a week, after 24 to 36 hours in vitro, the “injured” cells that remain within each tissue culture dish are allowed to attach to the bottom of the dish, and the supernatant over the attached cells, including the associated “floating debris” are removed, discarded, and replaced with 10 ml of fresh sphere media. The media is changed once per week until floating spheres appear in each tissue culture dish. This is in contrast to the previous protocol in which the media was changed much more frequently.

Another improvement is the creation of floating spheres containing “stress reprogrammed” cells, by exposure of an aliquot of the cells to be reprogrammed, suspended in 100 ul of sphere media, without ATP, to a standard dose of electroporation to create small holes in the cells. This is done in the absence of the buffers that are normally added to the solution during standard electroporation to promote repair of the holes created in the cells, which in the case of “stress treatments”, is undesirable.

### IV. Methods of Making a Vaccine

The methods and compositions can be used in the development of cancer vaccines. Generating at least partially differentiated progeny of pluripotent tumor cells by treating tumor cells in accordance with the methods described herein can provide a diverse and changing antigen profile which can permit the development of more powerful APC (antigen presenting cells)-based cancer vaccines.

### V. Methods of Inducing an Immune Response to an Induced Pluripotent Cancer Cell; Disorders to be Treated

The vaccines produced from the CSCs are administered to a patient in need thereof. The vaccines cannot be administered to a patient that no longer has an intact immune system, since the vaccine needs to elicit a cellular and humoral response to the antigens on the CSCs to be effective. The vaccine may be the attenuated or killed CSCs, or components or antigens thereof. They may be administered with an adjuvant to enhance the immune response.

The vaccines are administered initially to “prime” the immune response, then the patient is reimmunized to insure as high a response to the vaccine as possible. Typically vaccine is administered at intervals of ten to 21 days for three to four doses. This may vary depending on concurrent therapy and the degree of integrity of the immune system.

The vaccine can be used to treat many different types of cancer, but the initial focus is on cancers for which there are no good therapeutic options, such as metastatic cancer, glioblastomas, pancreatic cancer and colon cancer, as well as drug resistant aggressive prostate and melanoma cancers. Glioblastoma is used as a representative type of cancer to demonstrate need for this type of therapy.

### Glioblastomas

Glioblastoma (GB) is the most frequent form of brain tumor in adults and is associated with a poor prognosis and a short median patient survival. Conventional theories state that cancer arises from an accumulation of somatic mutations, resulting in uncontrolled proliferation as well as selective growth advantage. Most commonly, cancer occurs in epithelial tissues. Whether a tumor originates from a differentiated cell, which regains the ability to proliferate, or whether it originates from a stem cell, which already has the capacity to proliferate, is not fully resolved, and depends on the tissue and the tumor itself. The existence of brain tumor propagating cells (BTPCs) and their molecular, genetic, and epigenetic footprint could open new ways of therapeutic approaches. In the last years, diverse tumors could be retraced to mutations in stem cells and various studies have suggested that NSCs might be the cells of origin of GB, including mutated astrocyte-like NSCs from the SVZ. Recent studies reported from clinics and mouse models that glioblastoma arise from migration of mutated astrocyte-like NSCs from the SVZ.

Glioma is an umbrella term, compromising around 30 percent of all brain tumors that are thought to grow from intrinsic glia cells. As an umbrella term glioma consolidates different types of tumors including ependymoma, astrocytoma, and oligodendroglioma, which vary in their symptoms, aggressiveness, malignancy, and treatment strategy. Glioblastoma multiforme (GB) belongs to the category of astrocytoma, is the most common and most aggressive of all malignant glial tumor in adults. Based on the World Health Organization classification, GB is the most malignant form of glioma and is classified as a grade IV tumor (ICD-O 9440/3) GB can be divided into primary (arising de novo) or secondary (developed from a pre-existing tumor) intrinsic brain tumor, however, 90% of all GB are primary. Specific mutations in the gene of isocitrate dehydrogenase (IDH) 1/2 are characteristic for secondary glioblastomas, which are more frequent in younger patients. High invasiveness of GB is recorded, with tumor cells mainly spreading into distinct brain regions, whereas metastasis into other organs is infrequent.

Diagnosis of GB comes with a poor prognosis with high morbidity and mortality. The median survival of patients diagnosed with GB and treated with the common medication is only 12 to 15 months. GB can occur in each age group; however, most of the patients are between 45-75 years old. Gliomas are mainly located in the cerebral cortex of adult brains, with 40% in the frontal lobe, followed by the temporal lobe (29%), the parietal lobe (14%), the occipital lobe (3%) and 14% of gliomas are positioned in deeper brain structures.

GBM presents unique challenges to therapy due to its location, aggressive biological behavior and diffuse infiltrative growth. Despite the development of new surgical and radiation techniques and the use of multiple antineoplastic drugs, a cure for malignant gliomas remains elusive. The scarce efficacy of current treatments reflects the resistance of glioblastoma cells to cytotoxic agents in vitro. Moreover, the short interval for tumor recurrence in glioblastoma patients suggests that tumorigenic cells are able to overtake the treatments without major damage.

The cancer stem cell (“CSC”) hypothesis asserts that solid tumors are maintained exclusively by a rare fraction of cancer cells with stem cell properties. The existence of cancer stem cells was first proven in the context of acute myeloid leukemia. More recently, this principle has also been extended to other tumors, such as breast and brain cancer. Cancer stem cells have been reported to be the only tumorigenic population in GBM, their unlimited proliferative potential being required for tumor development and maintenance. Thus, these cells should represent the primary therapeutic target in order to achieve complete eradication of the tumor. Eramo, et al. Cell Death & Differentiation 13, 1238-1241 (2006).

The mainstay treatment of GBM involves surgery, concurrent radiation with chemotherapy, and adjuvant chemotherapy with Temozolomide (TMZ; brand names Temodar and Temodal and Temcad) is an oral chemotherapy drug. It is an alkylating agent used as a treatment of some brain cancers; as a second-line treatment for astrocytoma and a first-line treatment for glioblastoma multiforme. Despite advances in the field, the overall survival rate remains only 15-19 months. The high degree of tumor heterogeneity in GBM contributes to treatment failure, to which functional and molecular heterogeneity and aberrant receptor tyrosine kinase (RTK) activity all contribute. CSCs located at the top of the hierarchy initiate and maintain the tumor after treatment. Glioma CSCs have also been shown to contribute to radiation resistance by increasing the DNA damage response machinery. In terms of molecular heterogeneity, different subtypes of GBM with distinct molecular profiles coexist within the same tumor and likely exhibit differential therapeutic responses. A single-cell analysis of primary GBM patients showed that cells from the same tumor have differential expression of genes involved in oncogenic signaling, proliferation, immune response, and hypoxia. Furthermore, an increase in tumor heterogeneity was associated with a decrease in patient survival. A number of molecular mechanisms have been identified that mediate the therapeutic resistance of CSCs to cytotoxic therapies, including the DNA damage checkpoint, Notch, NF-κB, EZH2, and PARP, which suggests that CSCs develop multiple mechanisms of resistance that may require combinations of targeted agents.

Conventional treatment for GBM promotes a transient elimination of the tumor and is almost always followed by tumor recurrence, possibly with an increase in the percentage of CSCs, as CSCs are involved in tumor recurrence and therapeutic resistance. To effectively eliminate CSCs, it is critical to target their essential functions and their interactions with the microenvironment. Treatment with TMZ may kill CSCs that contain higher expression of the DNA repair protein MGMT; however, TMZ cannot prevent self-renewal of CSCs that contain MGMT. Another feature of CSCs is their ability to evade apoptosis. GBMs thrive in harsh microenvironments characterized by hypoxia and limited nutrient availability.

GBM may occur de novo in multiple types of neuro-epithelial cells, which is diagnosed as primary GBM, or it may arise following the progression or recurrence of low-grade glioma (LGG) into high grade form (HGG), in which case it is diagnosed as secondary GBM. Primary GBM is more prevalent, confers worse prognosis, and is understood to develop from distinct genetic precursors compared to secondary GBM. In addition to the distinction between primary and secondary GBM, malignant gliomas represent the most common mortality and morbidity among pediatric cancers. Especially, high grade gliomas that affect the midline structure of the brain [diffuse midline gliomas (DMG)] are among the poorest responders to existing treatments, due in part to the unique genetic and epigenetic mechanisms driving the development of these tumors. The wide differences in tumor etiology and genetic landscape among GBM necessitate different treatment approaches and have resulted in a patient population with an acute need for improved therapy.

The current standard of care involves maximal safe tumor resection followed by radiotherapy and chemotherapy. Despite advances in cytotoxic therapy regimens, targeted angiogenesis inhibitors and novel therapeutic modalities, such as alternating electric field therapy, patient survival has only improved modestly over recent years. Immunotherapy is an emerging therapeutic approach for GBM. The central nervous system (CNS) was once considered an immune privileged site that was spared from the potentially damaging effects of active immune responses. However, decades of research into the role of the immune system within the CNS has amended this preconception and allowed for a deeper understanding of how the adaptive immune response can function in the CNS. Recent studies investigating peptide vaccines and adoptive cell transfer for patients with malignant glioma have demonstrated that systemically administered treatments can, in fact, elicit antigen-specific T-cell responses. Despite these encouraging data, however, therapeutic responses were observed infrequently and had variable durations. The results of these initial trials underscore the need for continued in-depth research and analysis of the immunotherapeutic approaches for the treatment of glioma patients.

The development of vaccines based on heat-shock proteins, EGFRvIII (Del Vecchio et al. 2012), and DCs (Terasaki et al. 2011) has shown promising results in clinical trials. ICT-107, a patient-derived DC vaccine developed against six antigens highly expressed in glioma CSCs (Phuphanich et al. 2013), is currently under clinical evaluation for use in patients. Some of the challenges of developing therapeutic targeting agents are derived from the lack of universally informative markers to identify CSCs and the common molecular pathways shared by CSCs and NSPCs. The understanding of the biology of the CSCs and how these cells interact with their microenvironment in combination with the genetic and epigenetic landscape in GBM will be essential to develop more effective therapies. See Lathia, et al. Genes Dev. 2015 Jun. 15; 29(12): 1203-1217.

Neural stem cells (NSCs), a subpopulation of astroglial cells, are self-renewing cells with the capacity to differentiate into multiple neural cell types like neurons and glial cells (astrocytes and oligodendrocytes). During development, NSCs are obligatory for the formation of the nervous system. They are most active in this period; however, since 1992 it is described that NSCs can also be found in the adult brain. Here, small populations of NSCs are located in specific stem cell niches that divide occasionally to generate differentiated cells including neurons (neurogenesis) and glial cells (gliogenesis).

The transformation of a cell into a tumorigenic cell includes multiple mutations. There are two prominent theories about the origin of cancer cells. The first theory about the origin of CSCs states that any body cell can become a cancer stem cell by mutation, meaning that already differentiated, somatic cells become tumorigenic. Therefore, an accumulation of mutations is needed in oncogenes (gain of function) or tumor suppressor genes (loss of function), which regulate cell growth, to transform somatic cells into CSCs. These mutations occur through replication errors or DNA damage, combined with a missing or incorrect repair mechanism. A second theory is called cancer stem cell theory. This theory is based on the self-renewal ability of stem cells or progenitor cells and states that CSCs arise through oncogenic mutation in stem cells. The idea of stem cells derived CSCs was minted by studies using human leukemia cancer cells, which were transferred into immunodeficient mice. When characterizing these cells, the authors found that the cells were quite heterogeneous and only a minor portion had the potential of producing leukemia in mice. This suggests that not all cancer cells but only the slowly dividing stem cells have the potential to reproduce the tumor itself. Another study addressed breast cancer cells and described the heterogeneous phenotype of the cells. Only a limited number of cells in the tumor displayed tumorigenic potential which they identified by cell surface markers (CD44⁺ CD24^{-/low} lineage⁻). Thus, targeting these cells by cancer therapy would be most promising.

In addition to their tumorigenic properties and extensive proliferative potential, CSCs share various qualities with normal stem cells: (I) The capacity of multipotency, meaning the ability to differentiate into multiple lineages, self-renewal, and the capacity to divide into either new stem cells or into differentiated cells. (II) A low self-renewal rate and rare occurrence (only one in a million cells). (III) A strict control by their microenvironment to regulate the balance between proliferation and cell death. (IV) The usage of similar signaling pathways. The hypothesis of CSCs can also be extended to brain tumors, here referred to as brain tumor propagating cells (BTPCs), however, with some minor deviations. As discussed above, stem cells are scarce in the adult brain and can only grow in protective stem cells niches, including the hippocampus and the SVZ. These NSCs already possess the ability to proliferate and thus they could transform more easily and rapidly into BTPCs than any other post-mitotic neural cell in the brain. After certain variations, neural precursor cells could become BTPCs. However, other than their offspring, NSCs normally do not leave their neurogenic niches. One hypothesis would be that BTPCs originate from a mutation or deregulation that enables the NSCs to migrate and leave the niche. This exit and a subsequent dysregulation of the stem cell might result in unpredictable proliferation and thus tumorigenesis. Due to specific BTPC characteristics, like slow cell division rate, self-renewal properties, high capacity for DNA repairing and high expression of drug transporters, the identification and targeting of this cell population represents a challenge to this day. Moreover, BTPCs are capable of developing resistance mechanisms in multiple ways complicating conventional drug efficacies. High expression of ATP-binding cassette drug transporters can impede cytotoxic agents to enter the cell, resulting in resistance to different chemotherapeutic drugs including the commonly used alkylating agent temozolomide and increasing the risk of tumor recurrence after the treatment. Besides the chemo-resistance, BTPCs are capable of developing a radio-resistance by an increase in the activation of the DNA repair machinery, which is promoted by the expression of stem cell marker CD133. This combined chemo- and radio-resistance hampers a successful treatment and therefore many patients require combinational therapeutic strategies to improve the survival.

Another way BTPCs escape especially surgery is by forming stem cell niches and using ultra-long membrane protrusions, tumor microtubes, which can be found in various brain tumors and can be used as migration routes for cells located in BTPCs niches scattered in the brain. The brain and especially brain tumors are always considered as extremely difficult for treatment, due to the blood-brain barrier (BBB). The BBB normally hinders harmful substances and toxins to enter the brain via different cellular and molecular components as well as divers transport systems. However, the location of the SVZ at the border to the lateral ventricle introduces a new aspect to the system, the CSF, which is secreted by the choroid plexus, forming the blood-cerebrospinal fluid barrier (CSFB). This barrier is functionally distinct and is not as tight as the BBB; most non-cellular substances can enter the CSF. A further approach to diminish the number of BTPCs and to erase the tumors origin is the induction of apoptosis. Apoptosis includes a complex signaling network and the evasion of this system is crucial for the stem cell survival as well as tumor development. Altmann, et al. Cancers (Basel). 2019 April; 11(4): 448.

We claim:

1. A cancer vaccine comprising stress induced de-differentiated pluripotent cancer cells, or the lysate thereof.

2. The cancer vaccine of claim 1 further comprising de-differentiated cancer stem cells.

3. The cancer vaccine of claim 1 further comprising differentiated cancer cells.

4. The cancer vaccine of claim 1 further comprising an excipient for administration of the cells by injection.

5. The cancer vaccine of claim 1 wherein the stress is induced using stressing agents selected from the group consisting of chemical injury by acid exposure, exposure to inflammasomes or ATP, mechanical injury, and combinations thereof.

6. The cancer vaccine of claim 5 wherein the stressing agents are selected from the group consisting of electroporation, ultrasonification, trituration, and agitation.

7. The cancer vaccine of claim 5 wherein the stressing agents are the combination of mechanical and chemical injury.

8. The cancer vaccine of claim 5 wherein dedifferentiation into pluripotent cancer cells is induced by agitation of cancer biopsy cells at 750 RPMs (or cycles per minute) for 30 minutes in sphere media with ATP in an amount that causes a low pH and activation of inflammasomes.

9. The cancer vaccine of claim 1 wherein the cancer cells are obtained from a single patient biopsy.

10. The cancer vaccine of claim 1 wherein the cancer cells are obtained from cancer cells pooled from multiple individuals.

11. The cancer vaccine of claim 1 wherein the cells are lysed to form the vaccine.

12. The cancer vaccine of claim 1 wherein the cells or lysate is formulated for subcutaneous or transdermal injection, optionally with an adjuvant.

13. The cancer vaccine wherein the cancer cells are obtained from carcinomas, sarcomas, leukemias, lymphomas and myelomas, or central nervous system cancers.

14. The cancer vaccine of claim 13 wherein the cancer is selected from the group consisting of adenocarcinoma, basal cell carcinoma, squamous cell carcinoma, transitional cell carcinoma, bone cancer, prostate cancer, melanomas, and glioblastomas.

15. A method of vaccinating an individual against a cancer comprising administering an effective amount of the cancer vaccine of claim 1 to induce an immune response to the antigens in the cancer vaccine

16. The method of claim 15 wherein the vaccine comprises cells.

17. The method of claim 15 wherein the vaccine comprises cell lysate.

18. A method of making the cancer vaccine of claim 1 comprising
exposing differentiated cancer cells from an individual, a tumor thereof, or a tumor in cell culture to an effective amount of a stress inducing agent to cause differentiated tumor cells to dedifferentiate into pluripotent or stem cells.

19. The method of claim 18 wherein the stress inducing agents are selected from the group consisting of chemical injury by acid exposure, exposure to inflammasomes or ATP, mechanical injury, and combinations thereof.

20. The method of claim 18 wherein the stressing agents are selected from the group consisting of electroporation, ultrasonification, trituration, and agitation.

21. The method of claim 18 wherein the stressing agents are the combination of mechanical and chemical injury.

22. The method of claim 18 wherein dedifferentiation into pluripotent cancer cells is induced by agitation of cancer biopsy cells at 750 RPMs (or cycles per minute) for 30 minutes in sphere media with ATP in an amount that causes a low pH and activation of inflammasomes.

23. A method of making a cancer vaccine comprising isolating or identifying the antigens present in the cell lysate of claim 1.

24. The method of claim 23 further comprising identifying one or more antigens present in the cell lysate of claim 1 and making antibodies, antibody fragments or humanized antibodies or antibody fragments to the antigen for use as a cancer therapy.
